(19) United States
(12) Patent Application Publication
(10) Pub. No.: US 2009/0204668 A1
(43) Pub. Date: Aug. 13, 2009

(54) System and process for distant pulse diagnosis

(76) Inventor: Sydney Furan Huang, Oakland, CA (US)

Correspondence Address: LEON E. JEW, Esq.;Suite 288, 5776 Stoneridge Mall Rd., Pleasanton, CA, 94588, US

(21) Appl. No.: 12/378,362

(22) Filed: Feb. 12, 2009

Related U.S. Application Data
(60) Provisional application No. 61/065,559, filed on Feb. 12, 2008.

(51) Int. Cl. A61B 5/02; G06F 15/16

(52) U.S. Cl. 709/203; 600/500

(57) ABSTRACT

An Internet based system enabling a practitioner user to interpret the pulse of a distant patient user over the Internet and to make a diagnosis and give a prescription to the patient user. The pulse is collected from the patient user using a pulse collector which records a series of movements into pulse data files. The practitioner interprets the pulse either by reviewing a standardized file or by physically touching a pulse transformer which transforms the pulse data files into pulsations.

### REFERENCE TO RELATED APPLICATION

The present application claims priority to the provisional Appl. Ser. No. 61/065,559 filed on Feb. 12, 2008, the entire content of which is hereby incorporated by reference.

### FIELD OF THE INVENTION

The present invention relates generally to e-commerce technology. In particular, the invention relates to a web-based medical consultation system through which a patient-user can be diagnosed by a distant doctor-user and get a prescription from the doctor-user.

### BACK GROUND OF THE INVENTION

TCM practice requires considerable diagnostic skills. A training period of years or decades is usually necessary for TCM practitioners to understand the full complexity of symptoms and dynamic balances. Modern practitioners in China often use a traditional system in combination with Western methods.

Following a macro philosophy of disease, traditional Chinese medicine (TCM) diagnostics are based on overall observation of human symptoms rather than micro level laboratory tests. There are four types of TCM diagnostic methods: (1) observing, (2) hearing and smelling, (3) asking about background, and (4) touching. The pulse-reading component of the touching examination is so important that Chinese patients may refer to going to the practitioner as “going to have my pulse felt”.

In medicine, a person's pulse is the throbbing of the arteries as an effect of the heart beat. It can be felt at the neck (carotid artery), at the wrist (radial artery), behind the knee (popliteal artery), on the inside of the elbow (brachial artery), near the ankle joint (posterior tibial artery), and a few other places by the arteries compressing against bone. Pressure waves move the artery walls, which are pliable; these waves are not caused by the forward movement of the blood. As the heart contracting, the blood is ejected into the aorta and the aorta stretches. At this point, the wave of distention (pulse wave) is pronounced but relatively slow-moving (3-6 m/s). As it travels towards the peripheral blood vessels, it gradually diminishes and becomes faster. In the large arterial branches, its velocity is 7-10 m/s; in the small arteries, it is 15-35 m/s. The pressure pulse is transmitted fifteen or more times more rapidly than the blood flow.

The term pulse is also used, although incorrectly, to denote the frequency of the heart beat, usually measured in beats per minute. In most people, the pulse is an accurate measure of heart rate. Under certain circumstances, including arrhythmias, some of the heart beats are ineffective, and the aorta is not stretched enough to create a palpable pressure wave. The pulse is too irregular and the heart rate can be (much) higher than the pulse rate. In this case, the heart rate should be determined by auscultation of the heart apex, in which case it is not the pulse. The pulse deficit (difference between heart beats and pulsations at the periphery) should be determined by simultaneous palpation at the radial artery and auscultation at the heart apex.

A normal pulse rate for a healthy adult, while resting, can range from 60 to 100 beats per minute (BPM). During sleep, it can drop to as low as 40 BPM; during strenuous exercise, it may rise as high as 200-220 BPM. Generally, pulse rates are higher in younger people. The resting heart rate for an infant is usually close to an adult's pulse rate during strenuous exercise.

FIG. 1 is a schematic diagram illustrating the traditional manner of pulse diagnosis in Chinese medicine. A practitioner uses his hand 80 to touch and feel the wrist 81 of a patient. The practitioner collects information from his feelings on his fingers. When palpating the carotid artery, the femoral artery or the brachial artery, the thumb may be used. However, the thumb has its own pulse which can interfere with detecting the patient's pulse at other points, where two or three fingers should be used. Fingers or thumb must be placed near an artery and pressed gently against a firm structure, usually a bone, in order to feel the pulse. In the context of this application, “practitioner” and “doctor” are synonyms and are interchangeably used.

An alternative way of finding the pulse rate is by palpating or listening to the heartbeat. This is most commonly done with the examiner's palm or through a stethoscope. Before the invention of the stethoscope examiners would press their ear directly to the chest.

Pulses are felt at the inside of the wrist where the hand and arm meet. More precisely the first pulse is at the wrist crease on the radial side of the medial aspect of the forearm over the radial artery. The other two are just proximal, toward the elbow, to the first.

In addition to 9 positions there are three depths. Together they total 9 places with valuable diagnostic information on each wrist. There are 28 qualities of pulse which a practitioner must be alert for. Different combinations of pulses also have diagnostic significance. The number of possibilities allows fine distinctions in interpreting the gathered information.

Pulse-reading is considered an art that demands assiduous study and innate talent. The skill of a physician who can tell a patient's lifetime medical history from pulse-reading is not common. But pulse-reading skill varies like skill with a musical instrument. Study and talent are both factors but their proportions differ from person to person. Contrary to myth, a practitioner need not have spent 20 years in apprenticeship in order to make productive use of pulse-reading.

There is a need to standardize the pulse-reading using electronic analog and digital devices. If the pulse data is recorded in digital form, a distant practitioner may make diagnosis by obtaining the information included in the pulse data.

What is desired is a mechanical/electronic device for collecting and recording pulse data from a patient and a system for transmitting the collected pulse data to a practitioner who diagnoses pursuant to the pulse data and other information transmitted from the patient via a computer network such as the Internet. Also desired is an electronic/mechanical device (“pulse transformer” or “pulse player”) to transform the pulse data back into physical vibrations of an artificial arm from which a practitioner may read the “pulse”.

### SUMMARY OF THE INVENTION

The present invention teaches an Internet based system and process for remote diagnosis and prescription services. In the preferred embodiment of the present invention, the system includes a server computer, at least one data base associated with the server computer for storing reference files, at least one user computer used by an advisee user, at least one user computer used by an advisor user, a software application that runs across the Internet, and a pulse collector communicatively coupled to the user computer used by the advisee user. The server computer and the user computers are all communicatively coupled to the Internet. The pulse collector records a series of pulsations of a human being's pulse and transforms them into one or more data files. The software application includes a first interface from which the advisee user can upload and submit the data files to the server computer. The server computer transforms the data files into a standardized pulse file and forwards the standardized pulse file along with one or more relevant reference files to the advisor user. The software application includes a second interface from which the advisor user can review the standardized pulse file and the relevant reference files and create a response to the advisee user. The server computer stores the response as a new reference file. The first interface includes means for the advisee user to enter and submit textual information, means for the advisee user to upload and submit video and audio data file, means for the advisee user to select an advisor user from a list of advisor users who are online, and means for the advisee user to select an advisor user from a list of advisor users who are offline but are available to respond in a defined period of time. The second interface includes means for the advisor user to create a response by amending a relevant reference received from the server computer.

In another preferred embodiment, a pulse imitator is coupled to the advisor user computer. The pulse imitator re-transforms the data files received from the server into a series of pulsations. The practitioner may touch the imitator and interpret the pulse by his own feeling. In this embodiment, the server computer does not transform the data files received from the advisee user computer into standardized pulse file. Rather, the server forwards the data file, along with a number of relevant reference files from the database, to the practitioner who, in turn, creates a response based on his reading of the imitated pulse on the pulse transformer, the brief description entered by the advisee user, and the relevant reference files. In other words, the practitioner, not the server, performs the function of interpreting the pulse of the advisee user.

### BRIEF DESCRIPTION OF THE DRAWINGS

FIG. 1 is a schematic diagram illustrating the traditional manner of pulse reading diagnosis in Chinese medicine;

FIG. 2 is a schematic diagram illustrating a system for the pulse data collection using an electronic device and for distant diagnosis;

FIG. 3 is a schematic block diagram further illustrating the system for distant Chinese Medicine Diagnosis according to the invention;

FIG. 4 is a block flow diagram further illustrating the process according to the invention;

FIG. 5 is a schematic block diagram illustrating an evaluation mechanism which can be incorporated in the DPD system according to the present invention; and

FIG. 6 is a schematic diagram illustrating another preferred embodiment of the present invention.

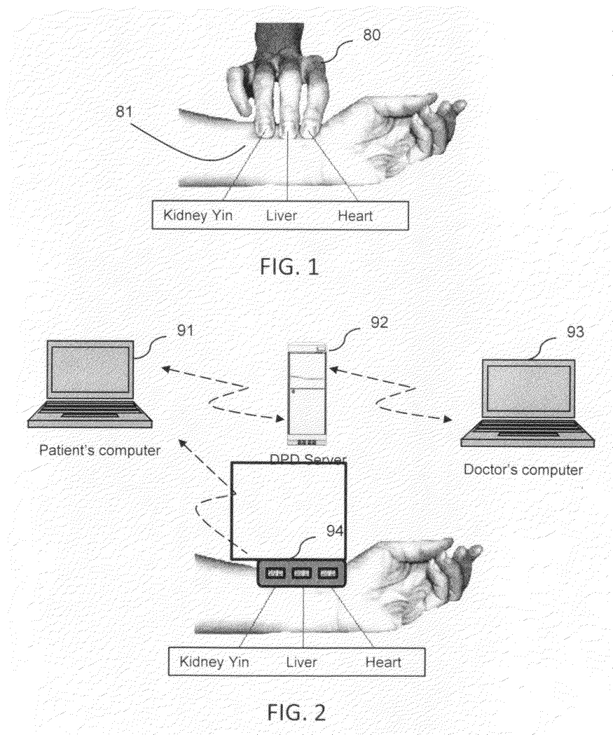

### DESCRIPTION OF THE INVENTION

FIG. 2 is a schematic diagram illustrating a system for the pulse data collection using an electronic device and for distant diagnosis. A user uses a pulse collector 94, which includes a number of sensors and electromagnetic recorders, to collect the pulse data and store it into digital format. It detects and records the pulse in compliance with the principles of the pulse diagnosis in Chinese medicine. In other words, the pulse collector faithfully records all subtle physical movements from which a practitioner may draw information for diagnosis purpose. The output of the pulse collector, called pulse file, is a digital file which can be transferred from the pulse collector to the advisee user computer and can be transferred via the Internet. The user logs in the website of Distant Pulse Diagnosis (DPD) server 92 via a computer 91 which is called advisee user computer, selects a distant practitioner from a list (optional), then uploads his pulse file which is the output of the pulse collector, and clicks “submit for diagnosis” in a graphical user interface. The interface is a component of the web-based software application which is hosted by the DPD server 92 and runs across the Internet. The DPD server 92 processes the pulse file received from the advisee user and transforms it into a standardized DPD pulse file. The standardized DPD pulse file includes a list of selected parameters and a brief description symptoms entered by the advisee user. The selected parameters are transformed and translated by the DPD server 92 from the pulse file. The DPD server 92 then attempts to match the standardized DPD pulse file to a pre-stored diagnosis (medical interpretation of the symptoms) and prescriptions in a database associated with the DPD server 92. The pre-stored diagnosis and prescriptions are called reference files. The database stores millions of reference files created by experienced practitioners. The DPD server 92 then forwards the DPD pulse file and the matching reference files, i.e., the relevant diagnosis-prescription files, to the selected practitioner for review and further diagnosis/prescription. If the advisee-user did not select a practitioner from a list, then the DPD server 92 would return the matching reference files and some instructional documents to the advisee-user for reference. If a practitioner is selected, the DPD server 92 notifies the practitioner by a standard e-mail or a pop-up message to the practitioner or other on-line means such as the Internet based automatic telephone. The practitioner, i.e., the advisor-user, logs in the system from the interface of the software application. By licking a link embedded in the notice, the practitioner may log in the system from the computer 93 which is called advisor user computer, review the advisee-user's DPD pulse file and the matching reference files (i.e., the relevant diagnosis-prescription files) provided by the DPD server 92, and write his own diagnosis-prescription report (“DPR”), which is also called response. After the advisor-user, which is also called as practitioner, submit his DPR response, the DPD server 92 stores it in the database for future reference, and then sends to the advisee-user a digital package including the practitioner's DPR response, other matching reference files (optional), and some notices (administrative information, such as warning and disclaimer notice, etc.)

In another preferred embodiment, the pulse collector 94 has multi-functions of (1) collecting the pulse data by detecting and recording the pulse in compliance with the principles of the pulse diagnosis in Chinese medicine, and (2) processing and transforming the data into a standardized DPD pulse file. Then, the user upload the file to the DPD server 92 which in turns matches the DPD pulse file to a pre-stored diagnosis (medical interpretation of the symptoms) and prescriptions in a database associated with the DPD server 92. The. DPD server 92 then forwards the “DPD pulse file” and the matching diagnosis-prescription reference files to the selected practitioner for review and further diagnosis/prescription.

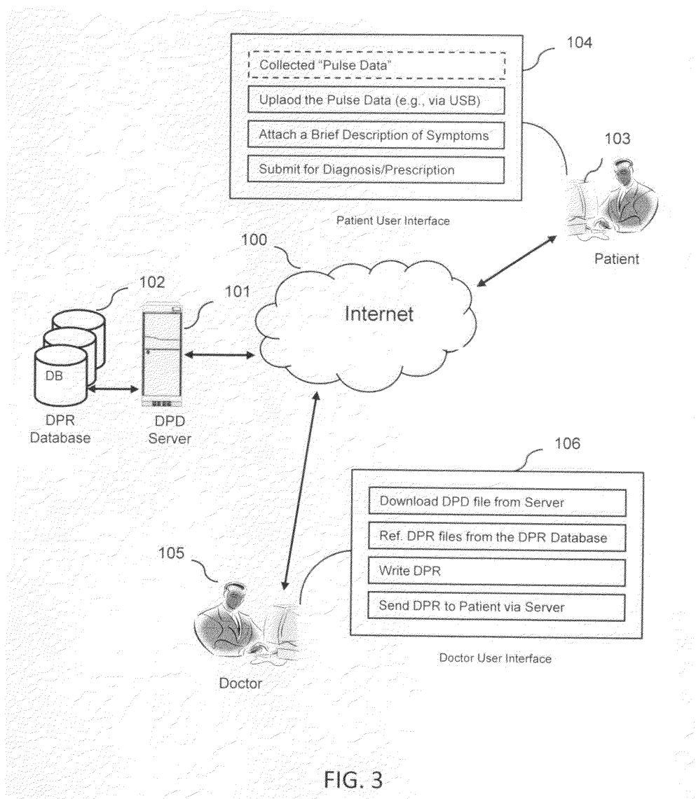

FIG. 3 is a schematic block diagram further illustrating the system for distant Chinese Medicine Diagnosis according to the invention. The system includes a central server computer 101 communicatively coupled to the Internet 100, a central database 102 associated with the central server 101, at least one patient end-user computer 103 and at least one practitioner end-user computer 105 communicatively coupled to the Internet 100. A number of software applications, which support the user interfaces described above, run across the server computer 101, the database 102, the user computers (103, 105) through the Internet 100. The patient user logs in the system via the user interface 104. The practitioner logs in the system via the user interface 106.

The user interface 104 includes: (1) the means of mouse-moving, clicking, dragging, etc., for enabling the user to identify and locate the “pulse file” from a USB or hard-drive associated with the user's computer; (2) the means of mouse-moving, clicking, dragging, etc., for enabling the user to upload the “pulse file”; (3) the means of mouse-moving, clicking, dragging, typing, etc., for enabling the user to add or attach a brief description of his symptoms; (4) the means for submitting his inquiry by clicking a virtual button in the interface; (5) the means for receiving response from the server/practitioner; and (6) the means for making feedback to the server or/and practitioner.

The patient-user can communicate with the practitioner-user online in real time. When both of them log on the system, they can see each other via the computer screen and can talk to each other via headsets. In an audio and video-enabled online chat room, the practitioner can make a diagnosis and a prescription based on (1) the patient's oral and/or written description of the symptoms, (2) the practitioner's observation of the patient-user's visual symptoms via the screen, and (3) the practitioner's hearing of the patient-user's audio symptoms via the speaker. However, empowered with the present invention, the practitioner can even deliberate and diagnose based on the pulse file provided by the patient-user.

Optionally, the system, via the interface 104, may provide the following options to the patient-user: (1) Do you want to upload a picture of your face? (2) Do you want to upload a close-up picture of your tongue? (3) Do you want to upload a close-up picture of your lips in natural closed position without any man-made products (e.g. lipstick, lip balm); and (4) do you want to enable the web-cam?

Optionally, the system, via the interface 104, may provide the following fields to ask for the patient-user's input:

Observing: (1) Describe your complexion (check all applicable fields); (2) Describe your tongue (check all applicable fields); (3) Describe your lips (check all applicable fields); (4) Describe your feces (check all applicable fields)

Smelling: (1) Describe your breath (check all applicable fields) and (2) Describe the smell of your feces (check all applicable fields).

Questioning: There is a list of general questions such as (1) How old are you? (choose an age group); (2) What is your regular occupation? (choose from some main selections); (3) How often do you exercise? (choose from some selections); (4) Describe your eating habit (check all applicable fields); (5) Do you have a pre-existing condition? (6) If yes on preexisting condition (check all applicable fields); (7) If no—have you consulted a medical practitioner regarding your systems? (8) Are your symptoms a result of a bodily injury? (9) Are your symptoms a result of a surgery? (10) Are you recently discharged from a hospital? (11) If yes, how long ago (choose from some main selections).

There is also a list of specific questions such as (1) Are you experiencing acute diarrhea? (2) If yes, how long have you been experiencing diarrhea? (3) Do you feel your symptoms could be a result of food poisoning? (4) If yes, stop here and consult a medical practitioner right away; (5) How do you feel? (check all applicable fields); (6) What kind of discomfort do you feel? (check all applicable fields); (7) Where do you feel the discomfort? (check all applicable fields); (8) What kind of pain do you feel? (check all applicable fields); (9) Where do you feel the pain? (check all applicable fields).

Pulse reading: Additional input section—limit to 100 words. Describe the key things that the patient-user felt having not been covered by the fields above. By clicking a virtual button, the patient-user may upload the DPD pulse file to the system.

For a patient user, what he needs to do includes: (1) use a pulse collector to get DPD pulse file; (2) connect the pulse collector to a web-enabled device such as desktop, laptop, PDA, and a cell-phone with a browser; (3) log on the DPD system via a user interface; (4) enter a brief description of his symptoms by clicking a number of answers to the pre-set questions, writing a brief message, and upload the DPD pulse file; (5) choose a practitioner from a list of on-line practitioners or a list of off-line practitioners; (6) choose whether or not enable the web-cam if it is available; (7) submit the inquiry. After the submission, the patient user will soon receive a response which may include a diagnosis and prescription report (DPR) by the practitioner elected by the patient-user, and a number of reference diagnosis-prescription reports (RDPRs). If the practitioner is online, the patient-user may chat with the practitioner for a while before the practitioner submits his DPR. If the patient-user trusts the DPR and has agreed the users' agreement, he may get the some herb from a nearby Chinese pharmacy and help himself.

The user interface 106 includes: (1) means for receiving and reviewing the “DPD pulse file” forwarded by the server; (2) means for receiving and reviewing certain server-provided reference diagnosis-prescription reports (RDPRs) for the similar “pulse symptoms”; (3) means for allowing the practitioner-user to write his own diagnosis-prescription report (DPR) or amend an existing reference diagnosis-prescription (RDPR); and (4) means for the user to send his prescription to the patient user via the server.

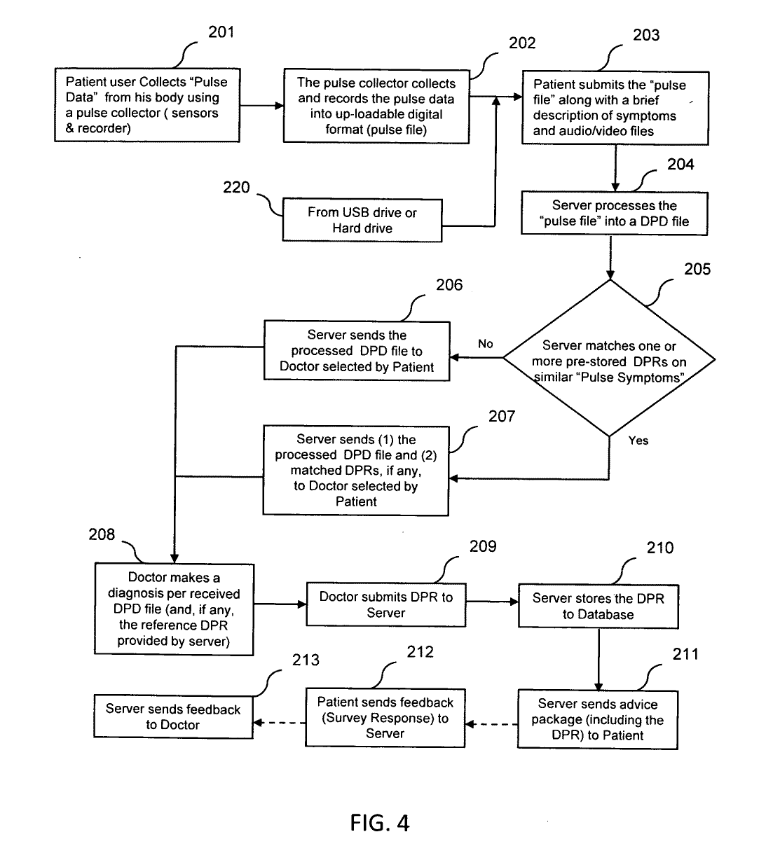

FIG. 4 is a block flow diagram further illustrating the process according to the invention.

Step 201: Using a pulse collector (also called a sensor), a patient-user collects pulse data from his body.

Step 202: The pulse collector collects and records the pulse data into up-loadable digital format (“source pulse file”).

Step 203: The patient-user uploads the “source pulse file” (Step 220) and submits it, along with a brief description of his symptoms, to the DPD server for advice.

Step 204: The DPD server processes the “source pulse file” into a standardized DPD file according to the attached information such as the brief description of symptoms and adjustment/calibrating parameters stored in the database.

Step 205: The DPD server then attempts to match the DPD file to the stored Diagnosis/Prescription Report (DPR) which include similar or substantially same symptom parameters as the new DPD file's. The format of a stored DPR includes three sections: symptoms, diagnosis, and the prescription. The format of a new DPD file is substantially same as the stored DPRs. The only difference is that the “diagnosis” section and the “prescription” section are blank in a new DPD file.

Step 206: If the matching is not successful, the server forwards the new DPD file to a practitioner selected by the patient user.

Step 207: If the matching is successful, the server forwards a number of matching DPRs and the new DPD file to the practitioner.

Step 208: The practitioner review the new DPD file and the reference DPRs provided by the server, and writes his own DPR or amend an existing reference DPR.

Step 209: The practitioner submits his DPR to the server.

Step 210: The server stores the practitioner's DPR to the database.

Step 211: The server sends the patient-user an advice package (including the practitioner's DPR, and optionally the reference DPR from the server's database).

Step 212: The patient-user may sends feedback to the server.

Step 213: The server then sends feedback to the practitioner.

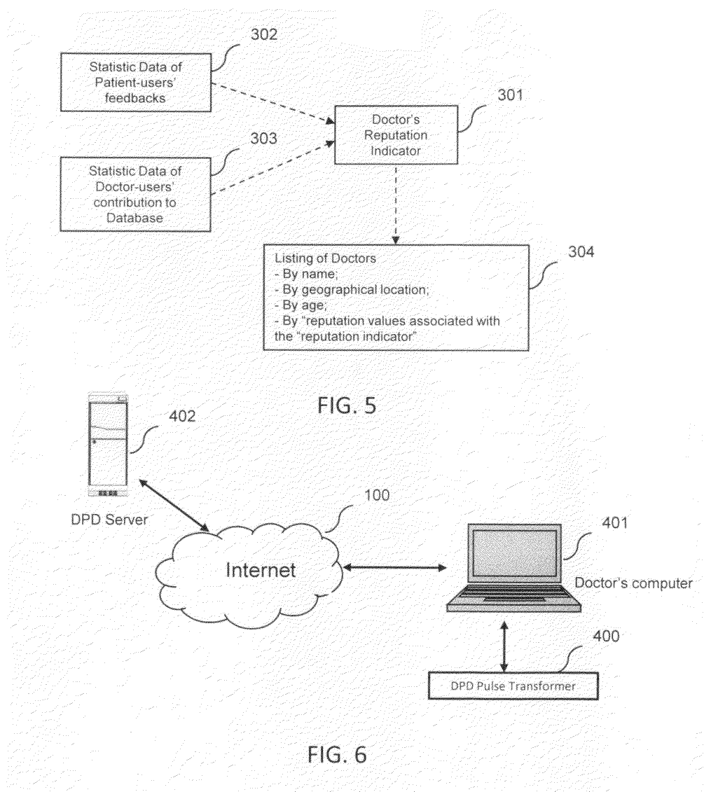

FIG. 5 is a schematic block diagram illustrating an evaluation mechanism which can be incorporated in the DPD system described above. The system maintains a database for practitioner's reputation index (data) 301 which is built according to the statistic data of patient-user's feedbacks 302 and the statistic data of practitioner-user's contribution to the DPR database 303. From the user interface (as shown in FIG. 3), the patient-user may select a practitioner from a list 303 according to the reputational index/reputation indicator.

FIG. 6 is a schematic diagram illustrating an additional feature of the invention. The system includes a hardware box 400, called pulse transformer, which is coupled to the practitioner-user's computer 401. When the practitioner receives a DPD file (the patient-user's pulse data), the practitioner may choose a function from the practitioner user interface to transform the DPD file back into imitated pulse which is performed by the electronic-mechanical device 400, i.e., the pulse transformer. The practitioner may actually touch the device 400 and feel the imitated pulse of a patient who is absent. In this way, a practitioner any where may make a diagnosis and prescribe for a patient who is anywhere else, as long as a DPD file can be made from the patient using the pulse collector, forwarding the DPD file to the practitioner, and the pulse transformer is available for the practitioner.

A typical application of this invention is that a company provides free-of-charge Chinese medical service by running an online system. The company recruits a number of qualified practitioners who may make diagnosis and prescription using the company's DPD interface and compensate the practitioners based on the quantity and quality of the services they provided. The patient user may buy or rent a pulse collector from the company. The company maintains a self-growing database of Chinese medicine prescriptions. Without using a pulse collector, a patient user may also get a diagnosis and prescription by entering his description of the symptoms. With the growing of the traffic of the users, the company makes revenues from selling online advertisements such as banners etc.

While one or more embodiments of the present invention have been illustrated above, the skilled artisan will appreciate that modifications and adoptions to those embodiments may be made without departing from the scope and spirit of the present invention.

1. An Internet based system for remote diagnosis and prescription, comprising:
a server computer;
at least one data base associated with the server computer for storing reference files;
at least one user computer used by an advisee user;
at least one user computer used by an advisor user;
a software application that runs across the Internet; and
a pulse collector communicatively coupled to the user computer used by the advisee user;
wherein the server computer and the user computers are communicatively coupled to the Internet;
wherein the pulse collector records a series of pulsations of a human being's pulse and transforms them into one or more data files;
wherein the software application comprises a first interface from which the advisee user can upload and submit the data files to the server computer;
wherein the server computer transforms the data files into a standardized pulse file and forwards the standardized pulse file along with one or more relevant reference files to the advisor user;
wherein the software application comprises a second interface from which the advisor user can review the standardized pulse file and the relevant reference files and create a response to the advisee user; and
wherein the server computer stores the response as a new reference file.

2. The system of claim 1, wherein the first interface comprises:
means for the advisee user to enter and submit textual information;
means for the advisee user to upload and submit video and audio data file;
means for the advisee user to select an advisor user from a list of advisor users who are online; and
means for the advisee user to select an advisor user from a list of advisor users who are offline but are available to respond in a defined period of time.

3. The system of claim 2, wherein the second interface comprises:
means for the advisor user to create a response by amending a relevant reference received from the server computer.

4. An Internet based system for remote diagnosis and prescription, comprising:
a server computer;
at least one data base associated with the server computer for storing reference files;
at least one user computer used by an advisee user;
at least one user computer used by an advisor user;
a software application that runs across the Internet; and
a pulse collector communicatively coupled to the user computer used by the advisee user;
a pulse transformer communicatively coupled to the user computer used by the advisor user;
wherein the server computer and the user computers are communicatively coupled to the Internet;
wherein the pulse collector records a series of pulsations of a human being and transforms them into one or more data files;
wherein the pulse imitator re-transforms the data files into a series of pulsations;
wherein the software application comprises a first interface from which the advisee user can upload and submit the data files to the server computer;
wherein the server computer forwards the data files along with one or more relevant reference files to the advisor user;
wherein the software application comprises a second interface from which the advisor user can activate the pulse imitator, review the relevant reference files and create a response to the advisee user; and
wherein the server computer stores the response as a new reference file.

5. The system of claim 4, wherein the first interface comprises:
means for the advisee user to enter and submit textual information;
means for the advisee user to upload and submit video and audio data file;
means for the advisee user to select an advisor user from a list of advisor users who are online; and
means for the advisee user to select an advisor user from a list of advisor users who are offline but are available to respond in a defined period of time.

6. The system of claim 5, wherein the second interface comprises: means for the advisor user to create a response by amending a relevant reference received from the server computer.

7. A process for providing remote diagnosis and prescription service via the Internet, comprising the steps of:
a. providing a pulse collector with which an advisee user collects pulse data from a human body, where in the pulse collector is communicatively coupled to a user computer;
b. providing means for recording the pulse data into up-loadable digital pulse file;
c. providing means for transferring the pulse file to a server computer;
d. providing means for processing the pulse file into a standardized file;
e. providing means for match the standardized file to relevant reference files in a database associated with the server computer;
f. if the matching is not successful, the forwarding the standardized file to a practitioner selected by advisee user;
g. if the matching is successful, then forwarding a number of matched relevant reference files along with the standardized file to the practitioner;
h. providing means for the practitioner to review the standardized file and, if any, the matched relevant reference files;
i. providing means for the practitioner to enter a response;
j. providing means for the server to store the response to the database; and
k. providing means for the advisee user to receive the response.

8. The process of claim 7, further comprises the steps of:
providing means for the advisee user to enter and submit textual information;
providing means for the advisee user to upload and submit video and audio data file;
providing means for the advisee user to select an advisor user from a list of advisor users who are online; and
providing means for the advisee user to select an advisor user from a list of advisor users who are offline but are available to respond in a defined period of time.

9. The process of claim 8, further comprises the steps of:
providing means for the advisor user to create a response by amending a relevant reference received from the server computer.
